(19) United States
(12) Patent Application Publication
(10) Pub. No.: US 2019/0231177 A1
(43) Pub. Date: Aug. 1, 2019

(54) FLEXIBLE IMAGING WINDOW

(71) Applicant: UVision360, Inc., Research Triangle Park, NC (US)

(72) Inventors: Erich Dreyer, Durham, NC (US); Allison London Brown, Durham, NC (US); David Robinson, Schaumburg, IL (US)

(21) Appl. No.: 15/885,105

(22) Filed: Jan. 31, 2018

(51) Int. Cl. A61B 1/00; A61B 1/018; A61B 1/05; A61B 1/07

(52) U.S. Cl. CPC A61B 1/00186; A61B 1/018; A61B 1/00137; A61B 1/07; A61B 1/05

(57) ABSTRACT

Various flexible windows and methods of use are provided herein that can be integrated with devices configured to image target tissue from a body cavity. In an exemplary embodiment, an imaging sleeve is provided with an elongate shaft having distal and proximal ends. The elongate shaft is configured to receive at least a portion of an imaging device therein, and an engagement member is disposed on the proximal end and configured to engage with the imaging device. A flexible window is disposed adjacent to the distal end and is configured to form to the imaging device to minimize distortion of light collected by the imaging device through the flexible window.

### FIELD

Devices, methods, and systems of imaging various target areas with a flexible window are provided herein.

### BACKGROUND

Minimally invasive surgeries, diagnostic procedures, exploratory procedures, and other medical procedures have been favored more and more by patients and physicians given the improved healing times and the less invasive nature of the operations. Various medical devices and instrumentation have been developed to accomplish these operations, such as medical introducers, imaging devices such as fiber optic scopes, and other related endoscopic devices.

For minimally invasive surgeries to be successful, however, imaging must be carried out on various target tissues and/or body cavities. Accurately and efficiently imaging tissue through a minimally invasive procedure, such as by using a fiber optic scope, can prove challenging for a variety of reasons, such as accessibility of the tissue to be imaged and distortion of any imaging conducted at the target site. For example, images of a target site can often be distorted through interference with light being collected by the imaging device. This distortion can be caused by the equipment used with the imaging device, such as by interference with a sleeve used to protect and deliver the imaging device to the target tissue.

Accordingly, there remains a need for improved devices, methods, and systems of imaging with a flexible window.

### SUMMARY

Devices, methods, and systems of imaging with a flexible window are provided herein. In one embodiment, an imaging sleeve is provided that includes an elongate shaft with distal and proximal ends. At least one lumen extends therebetween, and a longitudinal axis extends therealong. The at least one lumen is configured to receive at least a portion of an imaging device therein. An engagement member is positioned on the proximal end of the elongate shaft and is configured to engage with the imaging device. A flexible window is disposed adjacent to the distal end and is configured to form to the imaging device to minimize distortion of light collected by the imaging device therethrough.

The embodiment can have numerous variations. For example, a proximal-facing surface of the flexible window can be configured to sit flush against a distal end of the imaging device. In another example, the imaging sleeve can include a viewing lumen extending through the elongate shaft. The viewing lumen can be configured to receive the imaging device therein. A distal end of the viewing lumen can be sealed, and the proximal end of the elongate shaft can be open and configured to receive at least a portion of the imaging device therethrough. The distal end of the viewing lumen can have the flexible window disposed therein. The flexible window can also be configured to engage the elongate shaft through at least one of bonding and mechanical attachments. The imaging sleeve can also include a cap positioned on the distal end of the elongate shaft. The flexible window can be configured to engage the cap, and the cap can be configured to retain the flexible window in engagement with the imaging device. In one example, the flexible window can be formed through UV adhesion or a dip-molding. In another example, the flexible window can be configured to conform to one or more imaging devices configured to transfer images and/or video of visible light, electromagnetic radiation, microwaves, or ultraviolet radiation. The imaging sleeve can also include fluid inflow and fluid outflow lumens extending through the elongate shaft, which are respectively configured to deliver and remove fluid through the elongate shaft and to or from the distal end thereof. The fluid inflow and fluid outflow lumens can have openings at the proximal and distal ends of the elongate shaft. The imaging sleeve can also include a working lumen extending through the elongate shaft and configured to removably receive one or more surgical tools therethrough. The working lumen can have openings at each of the proximal and distal ends of the elongate shaft. In another example, the engagement member can be configured to prevent relative movement between the imaging sleeve and the imaging device.

In another aspect, an imaging system can be provided that has an imaging sleeve with an elongate shaft that has distal and proximal ends and at least one lumen extending therebetween. The imaging sleeve has an engagement member positioned on the proximal end of the elongate shaft, and the imaging sleeve has a flexible window positioned adjacent to the distal end of the elongate shaft that is configured to allow non-distorted imaging therethrough. The imaging system also has an imaging device with a handle and an elongate imaging member that extends distally from the handle. The elongate imaging member is configured to be inserted into the at least one lumen of the elongate shaft of the imaging sleeve, and the imaging device is configured to take images therefrom. The handle is configured to engage with the engagement member of the imaging sleeve. The flexible window and the elongate imaging member are also configured to engage flush with one another such that non-distorted imaging occurs through the flexible window.

The system can have numerous variations. For example, the elongate imaging member can have an imaging unit on a distal-most end thereof, and the flexible window can be configured to sit flush against the imaging unit. The imaging system can also include a viewing lumen that is configured to receive the elongate imaging system therethrough, and the flexible window can be positioned on a distal portion of the viewing lumen. The imaging system can have fluid in and fluid out lumens configured to deliver fluid to and remove fluid from the distal end of the elongate shaft. The imaging sleeve and the imaging device can be engaged by the engagement member such that the elongate imaging member has a distal-most position in the corresponding lumen of the imaging sleeve that is proximal to the flexible window of the elongate shaft. The engagement member can also be configured to prevent relative movement between the imaging sleeve and the imaging device. In one example, the imaging device can be a Complementary Metal-Oxide Semiconductor fiberscope.

In another aspect, a method of imaging a tissue sample includes positioning an imaging sleeve adjacent to tissue to be imaged by an imaging device disposed at least partially in the imaging sleeve. The imaging device acts to visualize the tissue through a flexible window on the imaging sleeve such that the flexible window forms to a distal end of the imaging device to reduce distortion of images taken thereby. The method also includes imaging a tissue sample using the imaging device positioned in engagement with the flexible window.

The method can vary in several ways. For example, the method can also include applying irrigation to a target tissue site by fluid in and fluid out lumens through the imaging sleeve. The method can also include passing a surgical tool through a working lumen of the imaging sleeve.

### BRIEF DESCRIPTION OF THE DRAWINGS

The invention will be more fully understood from the following detailed description taken in conjunction with the accompanying drawings, in which:

FIG. 1 is a side view of one embodiment of an imaging sleeve with an imaging device;

FIG. 2 is a side view of the imaging device of FIG. 1;

FIG. 3 is a side view of the imaging sleeve of FIG. 1;

FIG. 4 is a distal-to-proximal view of a distal tip of the imaging sleeve of FIG. 1;

FIG. 5 is a cross-sectional view of a distal end of another embodiment of an imaging sleeve with an imaging device and a flexible window;

FIG. 6 is a cross-sectional view of a distal end of another embodiment of an imaging sleeve with an imaging device and a flexible window;

FIG. 7 is a cross-sectional view of a distal end of another embodiment of an imaging sleeve with an imaging device and a flexible window;

FIG. 8 is a cross-sectional view of a distal end of another embodiment of an imaging sleeve with an imaging device and a flexible window; and

FIG. 9 is a cross-sectional view of a distal end of another embodiment of an imaging sleeve with an imaging device and a flexible window.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

When imaging tissue, especially in a non-invasive way, physicians are often required to access tissue that is difficult to reach and accurately image. This is especially true when the tissue in question is in a body cavity within a patient, such as when imaging uterine tissue. A sleeve and imaging device are thus often used to access body cavities and image tissue, as needed. However, the images obtained can be distorted due to a variety of factors, including obtaining the images through an image-distorting material. Various flexible windows are provided herein that can engage various sleeves and medical imaging devices that can be advanced into a patient and perform imaging of target tissue, thus providing users a way to accurately image remote tissue through minimally-invasive means. While various medical sleeves are described herein with reference to imaging devices, such as fiberscopes, that access uterine tissue, the flexible windows described herein can be used with a variety of types of imaging devices and can be used on a variety of target tissues or in other surgical applications.

In an exemplary embodiment, a sleeve is provided with an elongate shaft having distal and proximal ends. The elongate shaft is configured to receive at least a portion of an imaging device therein, and an engagement member is disposed on the proximal end and configured to selectively engage the imaging device. A flexible window is disposed adjacent to the distal end and is configured to conform to the imaging device to reduce or prevent distortion of light collected by the imaging device.

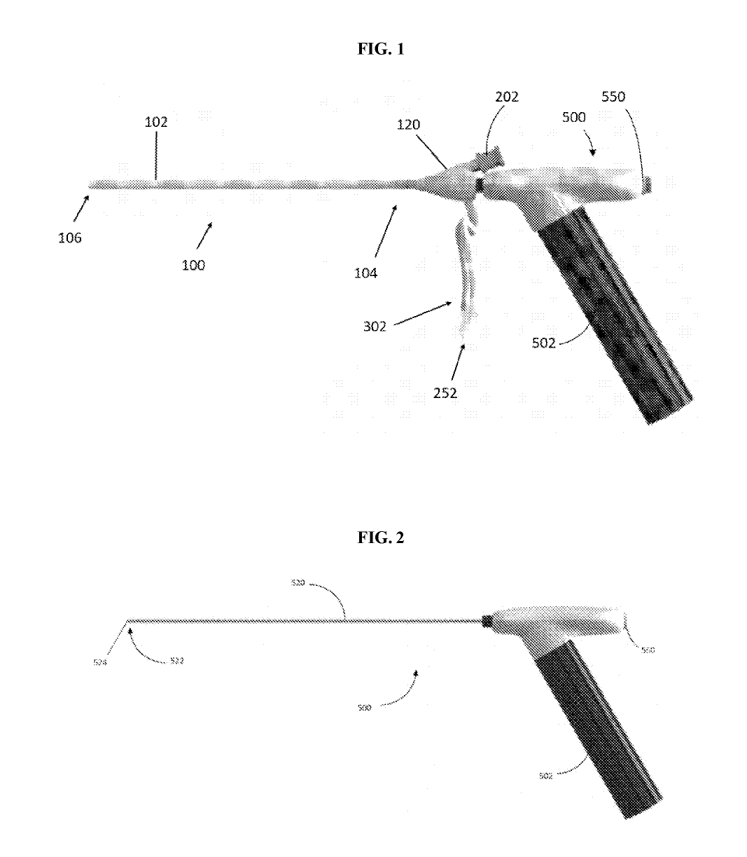

FIGS. 1-4 illustrate one embodiment of an imaging sleeve 100 with an imaging device 500. The imaging sleeve 100 includes a flexible elongate shaft 102 with an engagement member 120. The sleeve 100 can be engaged with the imaging device 500, similar to the devices illustrated in U.S. Patent App. Pub. No. 2017/0055813 to London Brown, et al., filed on Oct. 5, 2016, which is incorporated herein by reference in its entirety. The imaging device 500 can have a handle 502 and an elongate imaging member 520 extending distally from the handle 502. The handle 502 can include a light source disposed within the handle 502.

The elongate imaging member 520 extends from the handle 502 and is configured to be inserted through the engagement member 120 and into the elongate shaft 102. The elongate imaging member 520 has a distal portion 522 that terminates at a distal end of the elongate shaft 102 or at a point proximal to the distal end. The elongate imaging member 520 further includes an imaging unit 524 engaged on the distal portion 522 of the elongate imaging member 520. The imaging unit 524 can take a variety of forms, such as an active-pixel sensor array or a Complementary Metal-Oxide Semiconductor (CMOS) sensor, as provided in more detail in U.S. Patent App. Pub. No. 2017/0055813. The imaging unit 524 can be configured to be in communication with the handle 502 and can be configured to take images of areas adjacent thereto with assistance from a plurality of light transmission devices.

A plurality of light transmission devices extend from the light source in the handle 502, through a lumen defined by the elongate imaging member 520, and terminate at respective distal ends thereof disposed about the distal portion 522 of the elongate imaging member 520. The light transmission devices are typically arranged about the imaging unit 524. The light transmission devices are configured to receive the light from the light source and to transmit the light to the distal ends thereof to provide light to the area adjacent to the imaging unit 524 such that images can be taken within a body cavity of a patient. The light transmission devices can include fiber optic elements or light delivery fibers, and can be rigid or flexible to allow for bending or flexing within the elongate shaft 102.

The imaging device 500 can also be configured to connect to a power source. For example, a power source can be disposed in the handle 502, or the power source can be external to the device and connect to the handle 502. The power source can be arranged to be in electrical communication with the light source in the handle 502. A communication element 550 can also be operably engaged with the handle 502 such that the communication element 550 is in signal communication with the imaging unit 524 and configured to receive an image signal therefrom associated with an image captured by the imaging unit 524 or to communicate electrical power to the imaging unit 524. In addition, a display device for displaying the image, or a computer device for storing or analyzing the image, can be in communication with the communication element 550 via a wired communication arrangement or a wireless communication arrangement. All connections provided herein can be either wired or wireless connections.

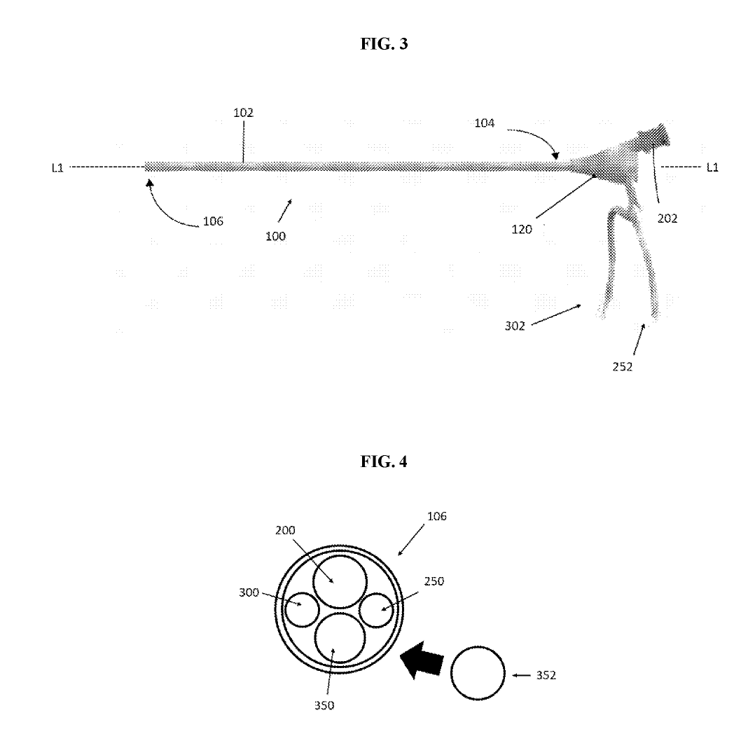

The elongate shaft 102 of the imaging sleeve 100 has the engagement member 120, a proximal end 104, a distal end 106, and one or more lumens that extend therethrough. As illustrated in FIGS. 3 and 4, four lumens 200, 250, 300, 350 extend therethrough, but any number of lumens can extend therethrough. Additionally, each lumen 200, 250, 300, 350 extends between the proximal end 104 and the distal end 106 of the elongate shaft 102. However, lumens can extend only partway between the proximal end 104 and the distal end 106. In some embodiments, the elongate shaft 102 can have an angled distal region that is angled at a non-zero angle relative to a longitudinal axis of the elongate shaft, such as being angled at approximately 15 degrees relative to the longitudinal axis of the elongate shaft.

The engagement member 120 is configured to engage the elongate shaft 102 on a distal end of the engagement member 120. The engagement member 120 is also configured to engage with the imaging device 500 on a proximal end of the engagement member 120. The engagement member 120 is configured to receive at least part of the imaging device 500 therethrough such that at least part of the imaging device 500 passes entirely therethrough and into a lumen of the elongate shaft 102. In some embodiments, the engagement member 120 is configured to secure the imaging device 500 with respect to the elongate shaft 102 such that the imaging device 500 is fixedly disposed in a distal position within the elongate shaft 102. As such, the imaging device 500 is non-slidable relative to and non-rotatable about a longitudinal axis L1 thereof within the elongate shaft 102. For example, the engagement member 102 can include one or more engagement fingers configured to engage corresponding features on the imaging device 500, such as in snap or friction fit engagements. The fingers can be configured to be engaged or disengaged by a user, such that the imaging sleeve 100 is removably, non-rotatably, and non-slidably affixed to the imaging device 500. Additionally, a variety of engagement features can be used, such as seals, clips, posts, locks, etc. While the sleeve 100 as illustrated is non-rotatable relative to the imaging device 500 when the engagement member is engaged, other embodiments can be provided with rotation means configured to rotate the sleeve 100 relative to the imaging device 500 without disengaging the sleeve 100 from the imaging device 500, for example by utilizing a rotation knob around the engagement member 120.

A working channel lumen 200 extends from the proximal end 104 to the distal end 106 and has openings on proximal and distal ends thereof. The working channel lumen 200 is configured to receive surgical instruments therethrough such that a surgical instrument can be inserted into the lumen 200 at the proximal end 104 so as to extend through and protrude from the distal end 106 of the elongate shaft 102. Surgical instruments can thus access, for example, a body cavity of a patient through the lumen 200. A distal end of the lumen 200 can be entirely open, allowing free passage of tools and materials therethrough. A proximal end of the lumen 200, however, can have various engagement mechanisms formed therein. For example, an engagement mechanism 202, such as a luer fitting, can be engaged with the proximal end of the lumen 200. The engagement mechanism 202 can be configured to engage, secure, and/or form a seal with the surgical instrument inserted into the working channel lumen 200.

Fluid inflow and fluid outflow lumens 250, 300 extend from the proximal end 104 to the distal end 106 of the elongate shaft 102 and have openings on proximal and distal ends thereof. The fluid inflow lumen 250 is configured to allow inflow of fluid to the body cavity, and the fluid outflow lumen 300 is configured to receive outflows of fluid from the body cavity. The fluid inflow lumen 250 engages an inflow irrigation tube 252, and the fluid outflow lumen 300 engages an outflow irrigation tube 302 to allow fluid to be provided and removed from the lumens 250, 300. One or more seals, engagements, mechanisms, valves, etc. can be disposed on proximal ends of the lumens 250, 300 to allow engagement with the tubes 252, 302, as explained further in U.S. Patent App. Pub. No. 2017/0055813 to London Brown, et al., filed on Oct. 5, 2016, which is incorporated herein by reference in its entirety.

A viewing lumen 350, which has an opening on the proximal end 104 of the elongate shaft 102, extends from the proximal end 104 to the distal end 106 of the elongate shaft 102. The viewing lumen 350 is configured to receive the imaging device 500 therein, and can be sealed at the distal end 106 of the elongate shaft, for example by a transparent member 352. The transparent member 352 is configured to allow imaging of the body cavity by the imaging device 500 therethrough and has an approximately circular shape. It extends across and seals only the viewing lumen, as illustrated in FIG. 3. However, it can have a variety of other configurations and extend across and seal a variety of other openings, as discussed below.

The transparent member 352 is an optically clear and flexible window that acts as both a protective sheath and a viral barrier for the imaging device 500 or any other viewing apparatus that requires an unobstructed view of a target subject. The member 352 is flexible, which allows the member 352 to conform to or flushly engage with the imaging unit 524 on the distal portion 522 of the elongate imaging member 520 such that a proximal or inward-facing surface 612 of the flexible transparent member 352 sits flat against the imaging unit 524, eliminating air or space between the two components. Such an engagement between the transparent member 352 and the imaging unit 524 is important so that the transfer or collection of light is not compromised when the imaging device 500 obtains images of the body cavity. Because the transparent member 352 sits against the imaging unit 524 and contours to the imaging unit 524 as needed, eliminating any excess space between the two components, a user can ensure that there is no distortion of the imaging performed by the imaging device 500. If the transparent member 352 was not able to flex or conform to the imaging unit 524, the collection of light by the imaging unit 524 might be compromised during imaging. The transparent member 352 can be disposed elsewhere along the elongate shaft 102, as long as the transparent member 352 is configured to conform to the imaging unit 524 as needed. The transparent member 352 can be made out of any optically clear and flexible material, such as silicone and/or various thermoplastic elastomers (TPE) and optically clear UV adhesives.

In use, a user engages the sleeve 100 with the imaging device 500 by inserting the elongate imaging member 520 into the elongate shaft 102 until the imaging unit 524 is positioned in a distal position and the engagement member 120 of the sleeve 100 engages with the handle 502 of the imaging device 500. The user inserts the distal end 106 of the sleeve 100 into a body cavity of a patient and positions the distal end 106 of the elongate shaft 102 adjacent to tissue to be imaged by the imaging device 500. The user can provide irrigation and/or cause suction by actuating the fluid flow paths of the fluid inflow and fluid outflow lumens 250, 300. The user can also pass various surgical tools down the working channel lumen 200 as required by the procedure being performed. When the procedure is completed and a user has obtained any desired images and performed any actions based on the desired surgical tools, the user removes the sleeve 100 from the body cavity, using images obtained from the imaging device 500 to guide removal as needed.

It should be noted that the optically clear and flexible window discussed herein is not limited to the described transparent member 352 and is not limited to use with the sleeve 100 and the imaging device 500. The flexible window provided herein can take a variety of forms and have a variety of configurations as needed such that it can conform to a corresponding viewing or imaging device to allow for the uncompromised transfer or collection of light. The flexible window can also be used on a variety of tools and in numerous situations, and not only on the end of a medical scope and to view a body cavity of a patient. Other uses can include use with devices configured to transfer images and/or video of visible light, other electromagnetic radiation, microwaves, ultraviolet radiation, and/or other wavelengths of light being transferred and viewed for diagnosis.

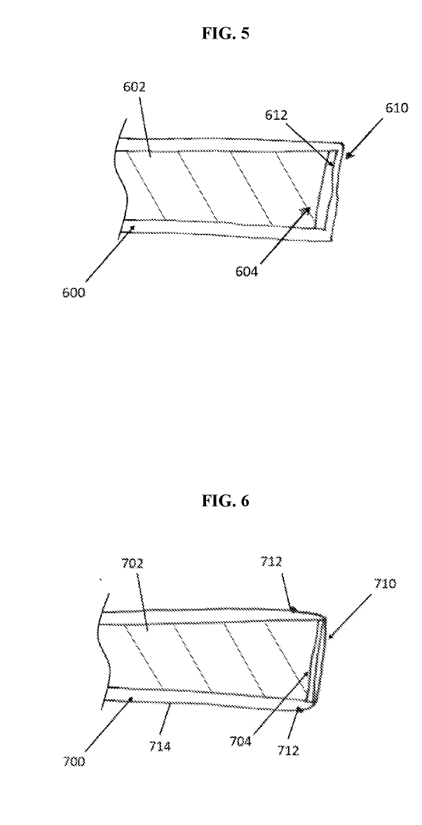

FIG. 5 illustrates an imaging device 600, similar to the imaging device 500, with an elongate shaft 602 and an imaging unit 604 disposed on a distal end thereof. The imaging unit 604 is similar to imaging unit 524, discussed above. A flexible window 610 is disposed over the distal end of the elongate shaft 602 and is configured to contour and flex such that the window 610 creates a sealed, protective sheath over the imaging unit 604, eliminating excess space between the two components to provide both a physical, protective barrier and a flush, optically-clear view therethrough for the imaging unit 604 of any desired target subject. The window 610 can cover the entire distal end of the elongate shaft 602, as illustrated here, or it can take the form of an isolated window that interacts with only the distal-most end of the elongate shaft 602 and the imaging unit 604 while integrating with a protective sheath that extends around the remaining portion of the elongate shaft 602.

FIG. 6 illustrates another embodiment of a flexible window 710 with an imaging device 700, similar to the imaging device 500, with an elongate shaft 702 and an imaging unit 704 disposed on a distal end thereof. The imaging unit 704 is similar to imaging unit 524, discussed above. The flexible window 710 is disposed over the distal-most end of the elongate shaft 702 and is configured to contour and flex such that the window 710 creates a sealed, protective sheath over the imaging unit 704, eliminating excess space between the two components to provide both a physical, protective barrier and a flush, optically-clear view therethrough for the imaging unit 704. The flexible window 710 engages a protective sheath 714 at one or more attachment points 712, such as through material bonding or mechanical engagements. The sheath 714 protects the remaining portion of the elongate shaft 702 and sealingly engages with the window 710 so that the elongate sheath 702 and the imaging unit 704 are entirely protected while the imaging unit 704 has an optically clear view through the flexible window 710 to any target area therebeyond. The attachment points 712 allows for the window 710 to be attached, removed, and replaced as needed by the user, such as if a different shape or configuration of flexible window is desired.

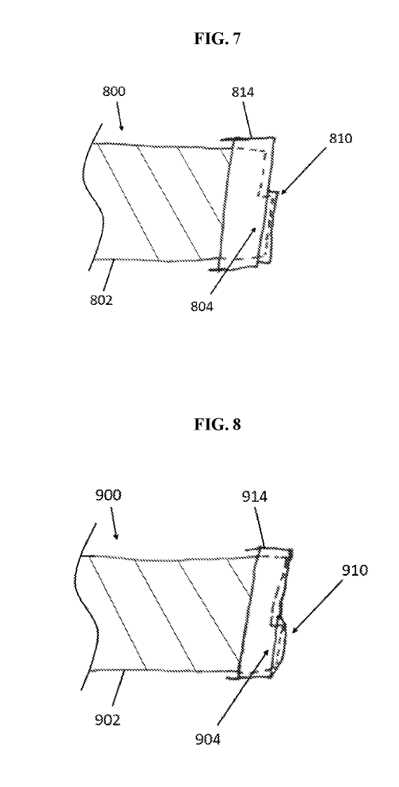

FIG. 7 illustrates another embodiment of a flexible window 810 with an imaging device 800, similar to the imaging device 500, having an elongate shaft 802 and an imaging unit 804 disposed on a distal end thereof. The imaging unit 804 is similar to imaging unit 524, discussed above. The flexible window 810 is disposed over the distal-most end of the elongate shaft 802 and is configured to contour and flex such that the window 810 creates a sealed, protective sheath over the imaging unit 804, eliminating excess space between the two components to provide both a physical, protective barrier and a flush, optically-clear view therethrough for the imaging unit 804. The flexible window 810 engages a cap 814 and can be integral therewith or can be affixed in place through one or more engagement means, such as through a friction fit or bonding means. The cap 814 engages the distal end of the elongate shaft 802 to provide protection thereto and secures the flexible window 810 in place over the imaging unit 804. The cap 814 can be made of a variety of materials, such as silicone or thermoplastic elastomers (TPE).

FIG. 8 illustrates another embodiment of a flexible window 910 with an imaging device 900, similar to the imaging device 500, having an elongate shaft 902 and an imaging unit 904 disposed on a distal end thereof. The imaging unit 904 can be similar to imaging unit 524, discussed above. The flexible window 910 is disposed over the distal-most end of the elongate shaft 902 and is configured to contour and flex such that the window 910 creates a sealed, protective sheath over the imaging unit 904, eliminating excess space between the two components to provide both a physical, protective barrier and a flush, optically-clear view therethrough for the imaging unit 904. The flexible window 910 is a UV adhesive window that engages a cap 914, similar to the cap 814. The cap 914 engages the distal end of the elongate shaft 902 to provide protection thereto and secure the flexible window 910 in place over the imaging unit 904. The cap 914 can be made of a variety of materials, such as silicone or thermoplastic elastomers (TPE).

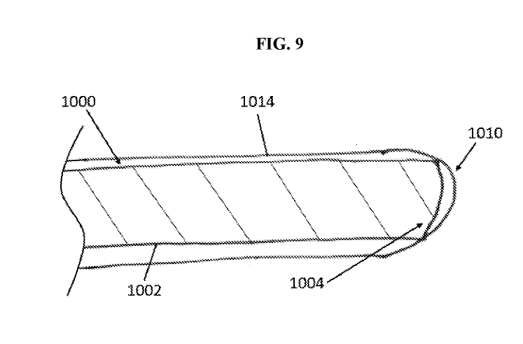

FIG. 9 illustrates another embodiment of a flexible window 1010 with an imaging device 1000, similar to the imaging device 500, having an elongate shaft 1002 and an imaging unit 1004 disposed on a distal end thereof. The imaging unit 1004 can be similar to imaging unit 524, discussed above. The flexible window 1010 is disposed over the distal-most end of the elongate shaft 1002 and is configured to contour and flex such that the window 1010 creates a sealed, protective sheath over the imaging unit 1004, eliminating excess space between the two components to provide both a physical, protective barrier and a flush, optically-clear view therethrough for the imaging unit 1004. The flexible window 1010 is a dip-molded window that can be integral with and/or can engage a cover or cap 1014 that itself engages the distal end of the elongate shaft 1002 to provide protection thereto. The window 1010 and the cap 1014 can each be made of a variety of materials, such as silicone or thermoplastic elastomers (TPE).

The flexible windows discussed herein can be constructed using various manufacturing processes, such as silicone injection molding, dip molding, UV adhesive, and/or mechanical construction. The flexible windows discussed herein can be used on a variety of devices and incorporated into a variety of surgical tools, such as the sleeve 100 and the imaging device 500 discussed above or on a variety of other tools. Additionally, the elongate shafts and the flexible windows can be made from a variety of different materials, as discussed above and including Polyether ether ketone (PEEK), High-density polyethylene (HDPE), Polytetrafluoroethylene (PTFE), Polyether block amide (PEBA) such as PEBAX, other thermoplastic elastomers (TPE), and/or Stainless Steel with any combination of polymers.

The sleeves disclosed herein can be provided in any of a variety of sizes, depending on patient anatomy, procedure type, imaging device to be used, and various other parameters which will be readily apparent to one having ordinary skill in the art. In some embodiments, the sleeves disclosed herein can have a variety of lengths, for example, about 10 cm to 40 cm, and can have a variety of diameters, such as about 1 mm to 6 mm.

In the present disclosure, like-numbered components of the embodiments generally have similar features, and thus within a particular embodiment each feature of each like-numbered component is not necessarily fully elaborated upon. Sizes and shapes of the devices described herein, and the components thereof, can depend at least on the anatomy of the subject in which the devices will be used, the size and shape of components with which the devices will be used, and the methods and procedures in which the devices will be used. The figures provided herein are not necessarily to scale. Although the devices and methods disclosed herein are generally directed to surgical techniques, they can also be used in applications outside of the surgical field. Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

1. An imaging sleeve, comprising:
an elongate shaft having distal and proximal ends, at least a viewing lumen and a working lumen extending therebetween within the elongate shaft, and a longitudinal axis extending therealong, the viewing lumen being configured to receive at least a portion of an imaging device therein, the working lumen being configured to receive a surgical tool therethrough;
an engagement member positioned on the proximal end of the elongate shaft and configured to selectively engage with the imaging device;
a flexible window sealing only a distal end of the viewing lumen and configured to conform to the imaging device to minimize distortion of light collected by the imaging device therethrough,
wherein a distal end of the working lumen is open to a surrounding environment adjacent to the distal end of the elongate shaft.

2. The imaging sleeve of claim 1, wherein a proximal-facing surface of the flexible window is configured to sit flush against a distal end of the imaging device.

3. (canceled)

4. The imaging sleeve of claim 3, wherein the proximal end of the elongate shaft is configured to receive at least a portion of the imaging device therethrough.

5. The imaging sleeve of claim 1, wherein the flexible window is configured to engage the elongate shaft through at least one of bonding and mechanical attachments.

6. The imaging sleeve of claim 1, further comprising a cap positioned on the distal end of the elongate shaft, wherein the flexible window is configured to engage the cap, and the cap is configured to retain the flexible window in engagement with the imaging device.

7. The imaging sleeve of claim 1, wherein the flexible window is formed through UV adhesion or a dip-molding.

8. The imaging sleeve of claim 1, wherein the flexible window is configured to form to one or more imaging devices configured to transfer images and/or video of visible light, electromagnetic radiation, microwaves, or ultraviolet radiation.

9. The imaging sleeve of claim 1, further comprising fluid inflow and fluid outflow lumens extending through the elongate shaft and configured to deliver and remove fluid through the elongate shaft and to or from the distal end thereof

10. (canceled)

11. The imaging sleeve of claim 1, wherein the engagement member is configured to prevent relative movement between the imaging sleeve and the imaging device.

12. An imaging system, comprising:
an imaging sleeve having distal and proximal ends and at least a first lumen and a second lumen extending therebetween within the imaging sleeve, the imaging sleeve having an engagement member positioned on the proximal end thereof, the first lumen of the imaging sleeve having a flexible window sealing only a distal end thereof and positioned adjacent to the distal end of the imaging sleeve, the flexible window being configured to allow non-distorted imaging therethrough, the second lumen being configured to allow a surgical instrument to pass therethrough from the proximal end of the imaging sleeve to extending distally beyond the distal end of the imaging sleeve; and
an imaging device including a handle and an elongate imaging member extending distally from the handle, the elongate imaging member being configured to be inserted into the first lumen of the imaging sleeve and the imaging device being configured to obtain images therefrom, the handle being configured to engage with the engagement member of the imaging sleeve,
wherein the flexible window and the elongate imaging member are configured to engage flush with one another to enable non-distorted imaging through the flexible window.

13. The imaging system of claim 12, wherein the elongate imaging member has an imaging unit on a distal-most end thereof such that the flexible window is configured to sit flush against the imaging unit.

14. (canceled)

15. The imaging system of claim 12, further comprising fluid in and fluid out lumens extending between the distal and proximal ends of the imaging sleeve and configured to deliver fluid to and remove fluid from the distal end of the imaging sleeve.

16. The imaging system of claim 12, wherein when the imaging sleeve and the imaging device are engaged by the engagement member, the elongate imaging member has a distal-most position in the first lumen of the imaging sleeve that is proximal to the flexible window of the imaging sleeve.

17. The imaging system of claim 12, wherein the imaging device is a Complementary Metal-Oxide Semiconductor fiberscope.

18. A method of imaging a tissue sample, comprising:
positioning an imaging sleeve adjacent to uterine tissue in a uterus of a patient to be imaged by an imaging device disposed at least partially in an imaging lumen within the imaging sleeve, the imaging device visualizing the uterine tissue through a flexible window sealing only a distal end of the imaging lumen in the imaging sleeve such that the flexible window forms to a distal end of the imaging device to reduce distortion of images taken thereby;
imaging a uterine tissue sample using the imaging device positioned in engagement with the flexible window; and
passing a surgical tool through a working lumen extending through the imaging sleeve to manipulate the uterine tissue sample disposed distally beyond the imaging sleeve such that a distal end of the working lumen is open to tissue surrounding the distal end of the imaging sleeve.

19. The method of claim 18, further comprising applying irrigation to a target uterine tissue site by fluid in and fluid out lumens extending through the imaging sleeve.

20. (canceled)

21. The imaging sleeve of claim 1, wherein a fluid seal is formed in the engagement member between a proximal end of the viewing lumen and the imaging device such that the viewing lumen is sealed from fluid and is configured to seal the portion of the imaging device received therein from fluid contact.

22. The imaging system of claim 12, wherein a fluid seal is formed between a proximal end of the first lumen of the imaging sleeve and the handle of the imaging device such that the first lumen is fluidly sealed on the proximal end and the distal end thereof and the imaging device is sealed from fluid contact from within the imaging sleeve.

23. An imaging system, comprising:
a disposable introducer having distal and proximal ends and at least a viewing lumen, a working lumen, and first and second fluid lumens extending therebetween, the introducer having an engagement member positioned on the proximal end thereof, the viewing lumen having a flexible window sealing only a distal end thereof such that distal ends of the working lumen and first and second fluid lumens are open to an external environment adjacent to the distal end of the introducer, the flexible window being configured to allow non-distorted imaging therethrough, the working lumen being configured to allow a surgical instrument to pass therethrough from the proximal end of the introducer to extend distally beyond the distal end of the introducer; and
a reusable imaging device including a handle and an elongate imaging member extending distally from and integral with the handle, the elongate imaging member being configured to be inserted into the viewing lumen of the introducer and the imaging device being configured to obtain images from a distal end thereof, the handle being configured to engage with the engagement member of the imaging sleeve,
wherein the flexible window and the elongate imaging member are configured to engage flush with one another to enable non-distorted imaging through the flexible window, and
wherein a fluid seal is formed between the viewing lumen and the imaging device such that the imaging device is fluidly separated from fluid within the introducer.
